(19) United States
(12) Patent Application Publication
(10) Pub. No.: US 2007/0190008 A1
(43) Pub. Date: Aug. 16, 2007

(54) Process for permanently reshaping the hair, comprising applying to the hair at least one precipitated fixing polymer, and multi-compartment device

(76) Inventors: Catherine Campain, Paris (FR); Priscille Devin-Baudoin, Vanves (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/641,722

(22) Filed: Dec. 20, 2006

Related U.S. Application Data
(60) Provisional application No. 60/771,445, filed on Feb. 9, 2006.

(30) Foreign Application Priority Data
Dec. 20, 2005 (FR) 05 12970

(51) Int. Cl. A61K 8/18

(52) U.S. Cl. 424/70.2; 424/70.4; 424/70.5; 424/70.51

(57) ABSTRACT

A process for permanently reshaping keratin fibers, comprising applying a reducing composition (A) comprising at least one salt chosen from organic and mineral salts-, shaping the keratin fibers by styling and applying a composition (B) comprising at least one fixing polymer; after a period of leave-on time, applying a fixing composition (C); and after a leave-on time, rinsing the keratin fibers; wherein the shaping by styling can be performed before, after or during the application of composition (B).

This application claims benefit of U.S. Provisional Application No. 60/771,445 filed Feb. 9, 2006, the contents of which are incorporated herein by reference. This application also claims benefit of priority under 35 U.S.C. § 119 to French Patent Application No. FR 05 12970, filed Dec. 20, 2005, the contents of which are also incorporated herein by reference.

The present disclosure relates to a process for permanently reshaping keratin fibers, comprising applying to the keratin fibers at least one precipitated fixing polymer.

Standard processes for permanently shaping keratin fibers such as the hair include a reduction step, comprising treating the keratin fibers with a reducing agent capable of cleaving the disulfide bridges of keratin, followed by a fixing step, comprising treating the keratin fibers with an oxidizing agent that re-establishes the disulfide bridges. These steps are performed while keeping the keratin fibers under a mechanical constraint, for example using curlers.

The traction exerted by the curlers on the hair makes it possible to obtain detachment of the roots, but also involves waving the hair. Now, many people wish to increase the volume of their head of hair without, however, having curly hair.

It is, admittedly, possible to use large-sized curlers to attenuate the curly appearance and simply obtain gentle waving, but the hold of such permanent reshapings is not always good.

Another drawback of the usual permanent-reshaping techniques lies in the frequent sensitization of the hair resulting from the repeated treatment of the hair with strong oxidizing and reducing agents used in large amounts.

To overcome this drawback, it has already been proposed to use gelled reducing compositions, which can allow the product to be localized on the roots and thus prevent chemical degradation of the hair over its entire length. However, the use of a gelled reducing composition does not dispense with the step of rolling up the hair on curlers. This technique consequently remains long and difficult to perform by the user and gives a reshaping result that is not very long-lasting.

A certain number of processes exist directed towards permanently reshaping the hair without using mechanical shaping means such as curlers. Thus, International Patent Application No. WO 99/18922 discloses a process for permanently reshaping the hair, without application of a tension constraint, which uses a thickened reducing composition that has moderate adhesive power, followed by an oxidizing composition, without intermediate rinsing intended to remove the reducing agent.

European Patent Application No. EP 1 064 921 describes a process for permanently reshaping the hair, in which, before applying the reducing composition, a separate and different composition comprising at least one anionic polymer is applied to the hair.

International Patent Application No. WO 01/95868 discloses a process for permanently reshaping the hair that is limited to certain areas, for example to the area close to the roots, which does not use mechanical means for placing the hair under tension. The hold of the shape given to the hair during the reduction and oxidation phases is ensured by the reducing composition itself, which contains a wax, absorbent hydrophilic particles, in particular particles of flour, and a reducing agent absorbed by the hydrophilic particles. This process is relatively complex, since it involves melting the wax in a suitable heating device and mixing the molten wax with the absorbent hydrophilic particles and the reducing agent. The application of the hot mixture to the hair requires a certain amount of dexterity on the part of the hair stylist and is difficult for the user to perform on him or herself. Moreover, relatively substantial stripping of the color can be observed when this technique is used for permanently reshaping dyed hair.

French Patent Application No. FR 2 858 551 discloses a process for permanently reshaping the hair, which is substantially easier to perform than that described in International Patent Application No. WO 01/95868, and which does not require prior preparation of the reducing composition by means of a heating device. The process described in French Patent Application No. 2 858 551 comprises, in order, the following steps:

(a) applying, to the areas to be reshaped, a solution of an anionic or amphoteric polymer in a medium containing water and, optionally, one or more cosmetically acceptable solvents,

(b) applying, to the areas treated in step (a), a reducing composition containing a reducing agent capable of cleaving the disulfide bridges of keratin and a sufficient amount of metal cations and/or of ammonium ions to make the anionic or amphoteric polymer precipitate in situ,

(c) shaping the hair thus treated, followed by a leave-on time of the reducing composition,

(d) applying, to the areas treated in steps (a) and (b), a fixing composition containing an oxidizing agent, followed by a period of leave-on time for the oxidizing composition, and

(e) rinsing the hair.

It has been observed that the effect obtained by means of this process is of only limited duration: the hold of the hair shape is not entirely satisfactory, especially with regard to repeated washing. Furthermore, the amount of fixing polymer required to implement this process makes this process expensive.

The problem to be solved by the present disclosure is directed towards a process for permanently reshaping keratin fibers, which does not require the use of mechanical tension means, which makes it possible to obtain long-lasting shaping of the keratin fibers, and which causes less sensitization of the keratin fibers than a standard permanent-waving operation.

The inventors have now discovered, surprisingly and unexpectedly, that it is possible to solve this problem by performing the process for permanently reshaping keratin fibers according to the present disclosure.

The present disclosure relates to the process for permanently reshaping keratin fibers, such as human keratin fibers and for example the hair, comprising, in the following order:

a) applying to the keratin fibers a reducing composition (A) comprising at least one keratin fiber reducing agent and at least one organic or mineral salt,

b) shaping the keratin fibers by styling and applying to the keratin fibers a composition (B) comprising at least one fixing polymer chosen from anionic and amphoteric fixing polymers, in an aqueous, alcoholic or aqueous-alcoholic medium,

c) after a period of leave-on time, application to the keratin fibers of a fixing composition (C) comprising at least one oxidizing agent, and

d) after a period of leave-on time, rinsing of the keratin fibers;

wherein the sub-steps of shaping the keratin fibers by styling and applying to the keratin fibers a composition (B) of step (b) can be performed in one of the following orders: shaping the keratin fibers by styling, followed by applying to the keratin fibers a composition (B); applying to the keratin fibers a composition (B), followed by shaping the keratin fibers by styling; or shaping the keratin fibers by styling and simultaneously applying to the keratin fibers a composition (B).

In at least one variant of this process, the sub-steps of step (b) are performed in the following order: shaping of the keratin fibers by styling, followed by application to the keratin fibers of a composition (B).

In another variant of this process, the sub-steps of step (b) are performed in the following order: application to the keratin fibers of a composition (B), followed by shaping of the keratin fibers by styling.

In yet another variant of this process, the sub-steps of step (b) are performed simultaneously.

In at least one other variant of this process, step (b) is followed by rinsing.

The process as disclosed herein also has the benefit of being selective, i.e. it may be performed on part of the hair: on only one area of the head, for example to correct a tuft, or on only part of the hair, for example at the roots.

The process as disclosed herein, allows hair shaping that lasts longer than the shaping obtained via the process disclosed in French Patent Application No. 2 858 551, without using a large amount of fixing polymer. At least one other benefit of the process according to the present disclosure is its economic advantage.

Other characteristics, aspects, subjects and benefits of the present disclosure will emerge even more clearly upon reading the description and the examples that follow.

The reducing composition (A) comprises at least one keratin fiber reducing agent and at least one salt chosen-from organic and mineral salts, the salts, for instance, being present in an amount that can lead to precipitating at least some or all of the at least one fixing polymer comprised in composition (B).

As used herein, the expression “fixing polymer in an aqueous, alcoholic or aqueous-alcoholic medium,” such as the at least one polymer used in the composition (B), is understood to mean that this polymer is in solution, in emulsion or in dispersion in the aqueous, alcoholic or aqueous-alcoholic medium used.

Without wishing to be bound by any theory, it is believed that, during the application of composition (B) comprising the at least one fixing polymer, the mixing—on the keratin fibers—of the reducing composition comprising a large amount of at least one salt chosen from organic and mineral salts, and of the composition comprising at least one fixing polymer allows a large increase in the ionic strength of the medium. Contraction of the macromolecular ball then takes place, finally resulting in precipitation of the at least one fixing polymer. Setting of the fiber or of the part of the fiber onto which the polymer has been deposited then takes place, making it possible to hold the hair in the desired shape during the reduction and fixing steps.

The ionic strength required to obtain the in situ precipitation of the at least one polymer depends on a number of factors, such as the number of charges of the polymer, the more or less hydrophobic nature of the polymer in its uncharged form, the molecular mass of the polymer, the concentration of the polymer in composition (B), or else the pH of the medium obtained by the in situ mixing of compositions (A) and (B).

This ionic strength may be provided by the at least one salt chosen from mineral and organic salts present in the reducing composition (A), but also by the reducing agent itself when it is in salt form.

To increase the ionic strength of the reduction medium to a sufficient value, any organic or mineral salt that does not significantly interfere with the reduction reaction of the disulfide bridges, for example ammonium salts or alkali metal salts, may, in principle, be used.

When the cations are provided by the reducing agent (in salt form), this salt comprises at least one carboxylate function. Non-limiting examples that may be mentioned include ammonium thioglycolate and ammonium thiolactate.

The at least one salt can be chosen from alkali metal salts, alkaline-earth metal salts and ammonium salts. Among the mineral salts that may be used, non-limiting mention may be made of alkali metal or alkaline-earth metal halides such as sodium chloride or magnesium chloride, and carbonates such as ammonium carbonate. The organic salts may be, for example, ammonium citrate.

The reduction step of the process of the present disclosure may be performed at a pH ranging from 7 to 11, for example, ranging from 7.5 to 9.5.

Of course, a person skilled in the art will know how to select, by virtue of his general technical knowledge, the pH modifiers and buffer agents that are suitable for adjusting the pH of compositions (A) and (B) to an appropriate value.

The inventors have found that the at least one salt chosen from organic and mineral salts concentration in the applied reducing composition (composition (A)) of greater than 0.3 M (mol/l) is sufficient to obtain a deposit of precipitated polymers of satisfactory quality allowing hold of the head of hair during the styling and oxidation steps. The concentration of the at least one salt chosen from organic and mineral salts concentration can range, for example, from 0.5 to 6 M.

The at least one reducing agent of the reducing composition be any known keratin-reducing agent, chosen from those usually used in the field of the permanent reshaping of the hair, such as sulfites, bisulfites and thiols. Among these reducing agents, non-limiting mention may be made of cysteine, cysteamine, thiolactic acid, thioglycolic acid and cosmetically acceptable salts thereof, for example, ammonium thioglycolate.

The at least one reducing agent can be present in the reducing composition in an amount ranging from 1% to 25% by weight, for example, an amount ranging from 1% to 10% by weight.

The reducing agent may be left to act for a time sufficient to obtain the desired reduction. This period of leave-on time ranges, for example, from 2 to 30 minutes and further, for example, from 5 to 15 minutes.

In one embodiment of the present disclosure, the head of hair may be shaped directly without leaving the reducing agent to stand thereon.

Any natural or synthetic fixing polymer that can give the head of hair a shape or modify the shape of the said head of hair may, in principle, be used for the implementation of composition (B). For example, the at least one fixing polymer used can be chosen from anionic and amphoteric polymers.

The anionic polymers that can be used in composition (B) are polymers comprising at least one group derived from carboxylic acids, sulfonic acids and phosphoric acids, and having a weight-average molecular mass ranging from 500 to 5,000,000.

The carboxylic groups are provided by unsaturated monocarboxylic or dicarboxylic acid monomers such as those of formula: 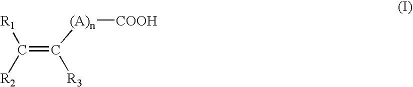
wherein n is an integer ranging from 0 to 10, A is a methylene group, optionally connected to the carbon atom of the unsaturated group or to the neighbouring methylene group when n is greater than 1 via a hetero atom such as oxygen or sulfur, R₁ is chosen from a hydrogen atom, and phenyl and benzyl groups, R₂ is chosen from a hydrogen atom and lower alkyl and carboxyl groups, and R₃ is chosen from a hydrogen atom, lower alkyl groups, and/or, —CH₂—COOH, phenyl and/or benzyl groups.

In formula (I) above, a lower alkyl group can comprise from 1 to 4 carbon -atoms, for example, methyl and ethyl groups.

Non-limiting examples of the anionic polymers comprising at least one group chosen from carboxylic and sulfonic groups that may be used are:

A) homo- or copolymers of acrylic or methacrylic acid or salts thereof, including copolymers of acrylic acid and of acrylamide and methacrylic acid/acrylic acid/ethyl acrylate/methyl methacrylate copolymers, for example Amerhold DR 25 sold by the company Amerchol, and the sodium salts of polyhydroxycarboxylic acids. Non-limiting mention may also be made of methacrylic acid/ethyl acrylate copolymers, for instance in aqueous dispersion, such as Luviflex Soft and Luvimer MAE sold by the company BASF;

B) copolymers of acrylic or methacrylic acids with a monoethylenic monomer such as ethylene, styrene, vinyl esters and acrylic or methacrylic acid esters, optionally grafted onto a polyalkylene glycol such as polyethylene glycol and optionally crosslinked. Such polymers are described, for instance, in French Patent Application No.1 222 944 and German Patent Application No. 2 330 956, copolymers of this type comprising in their chain an optionally N-alkylated and/or hydroxyalkylated acrylamide unit, such as those described, for instance, in Luxembourg Patent Application Nos. 75370 and 75371. Non-limiting mention may also be made of copolymers of acrylic acid and of C₁-C₄ alkyl methacrylate;

C) crotonic acid-based copolymers, such as those comprising in their chain vinyl acetate or propionate units and optionally other monomers such as allylic or methallylic esters, vinyl ether or vinyl ester of a saturated, linear or branched carboxylic acid comprising a long hydrocarbon-based chain such as those comprising at least 5 carbon atoms, these polymers possibly being grafted and crosslinked, or alternatively a vinyl, allylic or methallylic ester of an α- or β-cyclic carboxylic acid. For instance, such polymers are described, inter alia, in French Patent Nos. 1 222 944,1 580 545, 2 265 782, 2 265 781,1 564 110 and 2 439 798. Non-limiting examples of commercial products falling within this category are the resins 28-29-30, 26-13-14 and 28-13-10 sold by the company National Starch.

Non-limiting mention of crotonic acid-based copolymers include crotonic acid/vinyl acetate/vinyl t-butylbenzoate terpolymers and, for example, Mexomer PW sold by the company Chimex;

D) polymers derived from maleic, fumaric or itaconic acid or anhydride with vinyl esters, vinyl ethers, vinyl halides, phenylvinyl derivatives or acrylic acid and esters thereof; these polymers may be esterified. Such polymers are described, for instance, in U.S. Pat. Nos. 2,047,398, 2,723,248 and 2,102,113 and Great Britain Patent No. 839 805, and may be sold under the names Gantrez® AN or ES by the company ISP.

Polymers also falling within this category are the copolymers of maleic, citraconic or itaconic anhydrides and of an allylic or methallylic ester optionally comprising an acrylamide or methacrylamide group, an α-olefin, acrylic or methacrylic esters, acrylic or methacrylic acid or vinylpyrrolidone in their chain, the anhydride functional groups being monoesterified or monoamidated. These polymers are described, for example, in French Patent Nos. 2 350 384 and 2 357 241;

E) polyacrylamides comprising carboxylate groups;

F) polymers comprising sulfonic groups. These polymers may be polymers comprising vinylsulfonic, styrenesulfonic, naphthalenesulfonic, acrylamidoalkylsulfonic or sulfoisophthalate units.

By way of non-limiting example, these polymers may be chosen from:

polyvinylsulfonic acid salts with a molecular mass ranging from 1000 to 100,000, as well as copolymers with an unsaturated comonomer such as acrylic or methacrylic acids and esters thereof, as well as acrylamide or derivatives thereof, vinyl ethers and vinylpyrrolidone;

polystyrenesulfonic acid salts, sodium salts, having a molecular mass of 500,000 and of 100,000; these compounds are described, for example, in French Patent No. 2 198 719;

polyacrylamidesulfonic acid salts such as those mentioned in, for instance, U.S. Pat. No. 4,128,631;

G) grafted anionic silicone polymers.

The grafted silicone polymers used may be, for example, chosen from polymers comprising a non-silicone organic skeleton grafted with monomers comprising a polysiloxane, polymers comprising a polysiloxane skeleton grafted with non-silicone organic monomers, and mixtures thereof.

As used herein, the terms “silicone” and “polysiloxane” are understood to mean any organosilicone polymer or oligomer having a linear or cyclic, branched or crosslinked structure of variable molecular weight, obtained by polymerization and/or polycondensation of suitably functionalized silanes, and comprising a repetition of main units wherein the silicon atoms are linked together by oxygen atoms (siloxane bonding ≡Si—O—Si≡), optionally substituted hydrocarbon-based groups being linked directly via a carbon atom to the said silicon atoms. Non-limiting examples of hydrocarbon-based groups are alkyl groups, such as C₁-C₁₀ alkyl groups, and for instance, methyl, fluoroalkyl groups, aryl groups and, further for instance phenyl, and alkenyl groups and even further, for example, vinyl; other types of groups which can be linked, either directly or via a hydrocarbon-based group, to the siloxane chain are, for example, hydrogen, halogens such as chlorine, bromine or fluorine, thiols, alkoxy groups, polyoxyalkylene (or polyether) groups and for example polyoxyethylene and/or polyoxypropylene groups, hydroxyl or hydroxyalkyl groups, substituted or unsubstituted amine groups, amide groups, acyloxy or acyloxyalkyl groups, hydroxyalkylamino or aminoalkyl groups, quaternary ammonium groups, amphoteric or betaine groups, anionic groups such as carboxylates, thioglycolates, sulfosuccinates, thiosulfates, phosphates and sulfates, needless to say this list not being limiting in any way (“organomodified” silicones).

As used herein, the term “polysiloxane macromer” is understood to mean any monomer comprising in its structure a polymer chain of the polysiloxane type.

The polymers comprising a non-silicone organic skeleton grafted with monomers comprising a polysiloxane, used according to the present disclosure, comprise an organic main chain formed from organic monomers not comprising silicone, onto which is grafted, within the said chain and also optionally on at least one of its ends, at least one polysiloxane macromer.

The non-silicone organic monomers constituting the main chain of the grafted silicone polymer can be chosen from free-radical-polymerizable monomers comprising ethylenic unsaturation, polycondensation-polymerizable monomers, such as those forming polyamides, polyesters or polyurethanes, and ring-opening monomers, such as those of the oxazoline or caprolactone type.

The polymers comprising a non-silicone organic skeleton grafted with monomers comprising a polysiloxane that may be used can be obtained according to any means known to those skilled in the art, for instance by reaction between (i) a starting polysiloxane macromer which is correctly functionalized on the polysiloxane chain and (ii) at least one non-silicone organic compound correctly functionalized with a functional group which can react with the at least one functional group borne by the silicone, forming a covalent bond; a classic example of such a reaction is the free-radical reaction between a vinyl group borne on one of the ends of the silicone with a double bond of a monomer comprising ethylenic unsaturation in the main chain.

The polymers comprising a non-silicone organic skeleton grafted with monomers comprising a polysiloxane, in accordance with the present disclosure, may be chosen from those described, for example, in U.S. Pat. Nos. 4,693,935, 4,728,571 and 4,972,037 and European Patent Application Nos. 0 412 704, 0 412 707, and 0 640 105, and International Patent No. 95/00578. These are copolymers obtained by free-radical polymerization starting with monomers comprising ethylenic unsaturation and silicone macromers comprising a terminal vinyl group, or alternatively copolymers obtained by reaction of a polyolefin comprising functionalized groups and a polysiloxane macromer having a terminal functional group which is reactive with the functionalized groups.

One example of a family of grafted silicone polymers which is suitable for carrying out the present disclosure comprises grafted silicone copolymers comprising:

a) an amount ranging from 0% to 98% by weight of at least one free-radical-polymerizable lipophilic monomer (A) of low polarity comprising ethylenic unsaturation;

b) an amount ranging from 1% to 98% by weight of at least one polar hydrophilic monomer (B) comprising ethylenic unsaturation, which is copolymerizable with the monomer(s) of the type (A);

c) an amount ranging from 0.01% to 50% by weight of at least one polysiloxane macromer (C) of general formula:
X(Y)ₙSi(R)₃₋ₘZₘ (II)

wherein:

X is a vinyl group which is copolymerizable with the monomers (A) and (B);

Y is a divalent bonding group;

R is chosen from a hydrogen, C₁-C₆ alkyl and/or alkoxy, and C₆-C₁₂ aryl groups;

Z is a monovalent polysiloxane unit with a number-average molecular weight of at least 500;

n is 0 or 1 and m is an integer-ranging from 1 to 3; the percentages being calculated relative to the total weight of the monomers (A), (B) and (C).

For instance, these polymers are described, along with processes for their preparation, in U.S. Pat. Nos. 4,963,935, 4,728,571 and 4,972,037 and in European Patent Application Nos. 0 412 704, 0 412 707 and 0 640 105. They have a number-average molecular weight, for example, ranging from 10,000 to 2,000,000 and, for example, a glass transition temperature Tg or a crystal melting point Tm of at least −20° C.

As examples of lipophilic monomers (A), non-limiting mention may be made of acrylic or methacrylic acid esters of C₁-C₁₈ alcohols; styrene; polystyrene macromers; vinyl acetate; vinyl propionate; α-methylstyrene; tert-butylstyrene; butadiene; cyclohexadiene; ethylene; propylene; vinyltoluene; acrylic or methacrylic acid esters of a 1,1-dihydroperfluoroalkanol or of homologues thereof; acrylic or methacrylic acid esters of an ω-hydridofluoroalkanol; acrylic or methacrylic acid esters of a fluoroalkylsulfoamido alcohol; acrylic or methacrylic acid esters of a fluoroalkyl alcohol; acrylic or methacrylic acid esters of a fluoroether alcohol; and mixtures thereof.

The monomers (A) may be, for example, chosen from the group comprising n-butyl methacrylate, isobutyl methacrylate, tert-butyl acrylate, tert-butyl methacrylate, 2-ethylhexyl methacrylate, methyl methacrylate, 2-(N-methylperfluorooctanesulfoamido)ethyl acrylate and 2-(N-butylperfluorooctane-sulfoamido)ethyl acrylate, and mixtures thereof.

As examples of polar monomers (B), non-limiting mention may be made of acrylic acid, methacrylic acid, N,N-dimethylacrylamide, dimethylaminoethyl methacrylate, quaternized dimethylaminoethyl methacrylate, (meth)acrylamide, N-t-butylacrylamide, maleic acid, maleic anhydride and hemiesters thereof, hydroxyalkyl (meth)acrylates, diallyldimethylammonium chloride, vinylpyrrolidone, vinyl ethers, maleimides, vinylpyridine, vinylimidazole, heterocyclic vinyl polar compounds, styrene sulfonate, allyl alcohol, vinyl alcohol and vinylcaprolactam, and mixtures thereof. The monomers (B) may be, for example, chosen from the group comprising acrylic acid, N,N-dimethylacrylamide, dimethylaminoethyl methacrylate, quaternized dimethylaminoethyl methacrylate and vinylpyrrolidone, and mixtures thereof.

As polar monomers (B), the anionic grafted silicone polymers used according to the disclosure comprise at least one anionic monomer.

As examples of polysiloxane macromers (C) of formula (I), non-limiting mention may be made of those corresponding to the general formula (III) below: 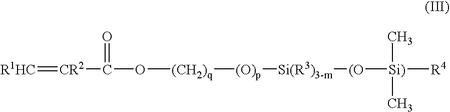
wherein:

R¹ is chosen from hydrogen and —COOH;

R² is chosen from hydrogen, methyl and —CH₂COOH;

R³ is chosen from C₁-C₆ alkyl, alkoxy, and alkylamino groups, and C₁-C₁₂ aryl and hydroxyl groups;

R⁴ is chosen from C₁-C₆ alkyl, alkoxy and alkylamino groups, and C₁-C₁₂ aryl and hydroxyl groups;

q is an integer ranging from 2 to 6;

p is 0 or 1;

r is an integer ranging from 5 to 700;

m is an integer ranging from 1 to 3.

The polysiloxane macromers of formula (IV): 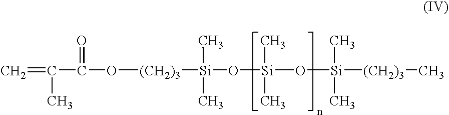
with n being an integer ranging from 5 to 700, may also be used.

In at least one embodiment of the disclosure, the copolymer that may be obtained by free-radical polymerization starting with the monomer mixture comprising of:

a) 60% by weight of tert-butyl acrylate;

b) 20% by weight of acrylic acid;

c) 20% by weight of the silicone macromer of formula (V): 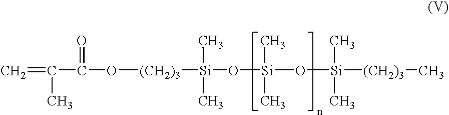
with n being an integer ranging from 5 to 700; the weight percentages being calculated relative to the total weight of the monomers.

Another family of silicone polymers that may be suitable for carrying out the present disclosure comprises grafted silicone copolymers which may be obtained by reactive extrusion-molding of a polysiloxane macromer with a reactive terminal functional group on a polymer of the polyolefin type comprising reactive groups that can react with the terminal functional group of -the polysiloxane macromer to form a covalent bond for grafting the silicone onto the main chain of the polyolefin.

For example, these polymers are described, along with a process for their preparation, in International Patent Application No. WO 95/00578.

The reactive polyolefins may be chosen from polyethylenes and polymers of ethylene-derived monomers such as propylene, styrene, alkylstyrene, butylene, butadiene, (meth)acrylates, vinyl esters or equivalents, comprising reactive functional groups that can react with the terminal function of the polysiloxane macromer. They may be, also, chosen from copolymers of ethylene or of ethylene derivatives and of monomers chosen from those comprising a carboxylic functional group such as (meth)acrylic acid; those comprising an acid anhydride functional group such as maleic anhydride; those comprising an acid chloride functional group such as (meth)acryloyl chloride; those comprising an ester functional group such as (meth)acrylic acid esters; and those comprising an isocyanate functional group.

The silicone macromers may be chosen from polysiloxanes comprising at least one functionalized group, at the end of the polysiloxane chain or close to the end of the chain, chosen from alcohols, thiols, epoxy groups and primary and secondary amines, and for example from those corresponding to the general formula (VI):
T-(CH₂)ₛ—Si—[—(OSiR⁵R⁶)ₜ—R⁷]_{y} (VI)
wherein T is chosen from NH₂, NHR′, epoxy, OH, and SH groups; R⁵, R⁶, R⁷ and R′ independently are chosen from C₁-C₆ alkyl, phenyl, benzyl, and C₆-C₁₂ alkylphenyl groups, and hydrogen; s is an integer ranging from 2 to 100; t is an integer ranging from 0 to 1,000 and y is an integer ranging from 1 to 3. They have a number-average molecular weight ranging from 5,000 to 300,000, for example from 8,000 to 200,000 and further, for example, from 9,000 to 40,000.

In another embodiment of the present disclosure, the grafted silicone polymer(s) comprising a polysiloxane skeleton grafted with non-silicone organic monomers comprising a silicone (or polysiloxane (≡Si—O—)ₙ) main chain onto which is grafted, within the chain and also optionally on at least one of its ends, at least one organic group not comprising silicone.

In, yet, another embodiment of the present disclosure, the polymers comprising a polysiloxane skeleton grafted with non-silicone organic monomers, may be existing commercial products or alternatively can be obtained according to any means known to those skilled in the art, for instance by reaction between (i) a starting silicone which is correctly functionalized on at least one of the silicon atoms, and (ii) a non-silicone organic compound which is itself correctly functionalized with a functional group that can react with the functional group(s) borne by the silicone, forming a covalent bond; a classic example of such a reaction is the hydrosilylation reaction between -Si-H groups and vinyl groups CH₂═CH—, or alternatively the reaction between thio-functional groups —SH with these same vinyl groups.

Non-limiting examples of polymers comprising a polysiloxane skeleton grafted with non-silicone organic monomers that may be suitable for carrying out the present disclosure, and also their specific mode of preparation, are described, for instance, in European Patent Application No.0 582 152, International Pat. Nos. WO 93/23009 and WO 95/03776, the teachings of which are incorporated in their entirety in the present disclosure by way of non-limiting references.

In another embodiment of the present disclosure, the silicone polymer comprising a polysilbxane skeleton grafted with non-silicone organic monomers which is used comprises the result of the free-radical copolymerization between, at least one non-silicone anionic organic monomer comprising ethylenic unsaturation and/or a non-silicone hydrophobic organic monomer comprising ethylenic unsaturation, and a silicone comprising in its chain at least one functional group that can react with the ethylenic unsaturations of the non-silicone monomers, forming a covalent bond, for example thio-functional groups.

In, yet, another embodiment of the present disclosure, the anionic monomers comprising ethylenic unsaturation may be chosen, alone or as mixtures, from linear or branched, unsaturated carboxylic acids, optionally partially or totally neutralized in the form of a salt, it being possible for this or these unsaturated carboxylic acids to be, for example, acrylic acid, methacrylic acid, maleic acid, maleic anhydride, itaconic acid, fumaric acid and crotonic acid. The suitable salts can be, for example, alkali metal salts, alkaline-earth metal salts and ammonium salts. It will likewise be noted that, in the final grafted silicone polymer, the organic group of anionic nature which comprises the result of the free-radical (homo)polymerization of at least one anionic monomer of unsaturated carboxylic acid type can, after reaction, be post-neutralized with a base (sodium hydroxide, aqueous ammonia, etc.) in order to place it in the form of a salt.

In, yet, another embodiment of the present disclosure, the hydrophobic monomers comprising ethylenic unsaturation may be chosen, alone or as mixtures, from acrylic acid esters of alkanols and/or methacrylic acid esters of alkanols. The alkanols are, for example, C₁-C₁₈ and further, for example, C₁-C₁₂. The at least one monomer may be, for example, chosen from isooctyl (meth)acrylate, isononyl (meth)acrylate, 2-ethylhexyl (meth)acrylate, lauryl (meth)acrylate, isopentyl (meth)acrylate, n-butyl (meth)acrylate, isobutyl (meth)acrylate, methyl (meth)acrylate, tert-butyl (meth)acrylate, tridecyl (meth)acrylate and stearyl (meth)acrylate.

One family of silicone polymers comprising a polysiloxane skeleton grafted with non-silicone organic monomers that is suitable for carrying out the present disclosure, are silicone polymers comprising in their structure at least one unit of formula (VII):

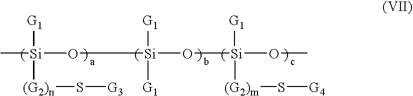

wherein the groups G₁, which may be identical or different, are chosen from a hydrogen, C₁-C₁₀ alkyl groups, and phenyl groups; the groups G₂, which may be identical or different, are chosen from C₁-C₁₀ alkyl groups; G₃ is chosen from polymer residues resulting from the (homo)polymerization of at least one anionic monomer comprising ethylenic unsaturation; G₄ is chosen from polymer residues resulting from the (homo)polymerization of at least one hydrophobic monomer comprising ethylenic unsaturation; m and n are equal to 0 or 1; a is an integer ranging from 0 to 50; b is an integer which may range from 10 to 350, c is an integer ranging from 0 to 50; with the proviso that one of the parameters a and c is other than 0.

The unit of formula (VII) above may have at least one of the following characteristics:
the groups G₁ can be an alkyl group, for example a methyl group;
n is non-zero, and the groups G₂ can be a divalent C₁-C₃ group, for example a propylene group;
G₃ can be a polymer group resulting from the (homo)polymerization of at least one monomer of the carboxylic acid type comprising ethylenic unsaturation, such as acrylic acid and/or methacrylic acid;
G₄ can be a polymer group resulting from the (homo)polymerization of at least one monomer of the (C₁-C₁₀)alkyl(meth)acrylate type, for example isobutyl or methyl(meth)acrylate.

Non-limiting examples of grafted silicone polymers corresponding to formula (IV) are, for example, polydimethylsiloxanes (PDMS) onto which are grafted, via a thiopropylene-type connecting chain, mixed polymer units of the poly(meth)acrylic acid type and of the polymethyl (meth)acrylate type.

The number-average molecular mass of the silicone polymers comprising a polysiloxane skeleton grafted with non-silicone organic monomers of the disclosure, ranges from 10,000 to 1,000,000 and for example ranges from 10,000 to 100,000.

Among the grafted silicone polymers may be used, non-limiting mention may be made of the product sold by the company 3M under the reference VS80.

H) anionic polyurethanes.

In another embodiment of the present disclosure, the polyurethanes may comprise a base repeating unit of formula (VIII):
—X′—B—X′—CO—NH—R—NH—CO— (VIII)
wherein
X′ is an O atom and/or NH,
B is a divalent hydrocarbon-based group, this group being substituted or unsubstituted, and
R is a divalent group chosen from branched and unbranched alkylene groups of C₆-C₂₀ aromatic, C₁-C₂₀ and for example C₁-C₆ aliphatic and C₁-C₂₀ and further, for example C₁-C₆ cycloaliphatic type, these groups being unsubstituted or substituted with at least one halogen, C₁-C₄ alkoxy or C₆-C₃₀ aryl, for example phenyl, groups.

The group B can be a C₁-C₃₀, for example a C₂-C₁₀ divalent group, and bears a group comprising at least one carboxylic functional group and/or at least one sulfonic functional group, the carboxylic and/or sulfonic functional groups being in free form or partially or totally neutralized with a mineral or organic base such as alkali metal or alkaline-earth metal hydroxides, aqueous ammonia, alkylamines, alkanolamines or organic amino acids. B may be, for example, the divalent group derived from dimethylolpropionic acid.

For example, the group R can be chosen from the groups corresponding to the following formulae: 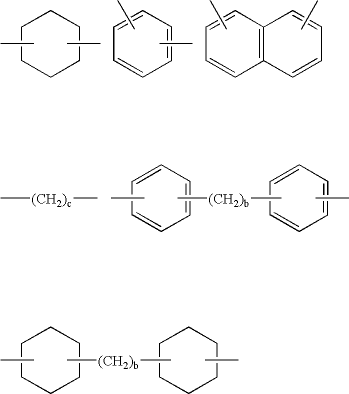
wherein b is an integer ranging from 0 to 3 and c is an integer ranging from 1 to 20, for instance ranging from 2 to 12.

In one embodiment of the present disclosure, the group R may be chosen from hexamethylene, 4,4′-biphenylenemethane, 2,4- and/or 2,6-tolylene, 1,5-naphthylene, p-phenylene and methylene-4,4-bis-cyclohexyl groups and the divalent group derived from isophorone.

The fixing polyurethanes that may be used may comprise silicone grafts and silicones comprising hydrocarbon-based grafts.

At least one polyurethane that can be used may also comprise at least one polysiloxane block, and its base repeating unit corresponds, for example, to the general formula (IX):
—X′—P—X′—CO—NH—R—NH—CO— (IX)
wherein:
P is a polysiloxane segment,
X′ is an O atom and/or NH, and
R is a divalent group chosen from branched and unbranched alkylene groups of C₆-C₂₀ aromatic, C₁-C₂₀, for example C₁-C₆ aliphatic and C₁-C₂₀ and further for example C₁-C₆ cycloaliphatic type, these groups being unsubstituted or substituted with at least one entity chosen from halogen, C₁-C₄ alkoxy and C₁-C₃₀ aryl groups, for example phenyl groups.

In another embodiment of the present disclosure, the group R may be chosen from the groups corresponding to the following formulae: 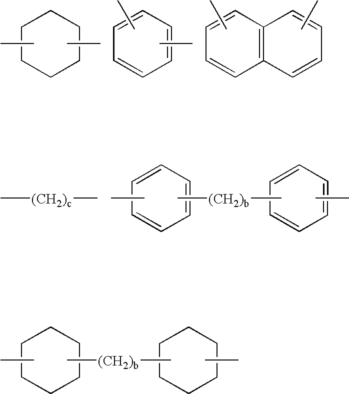
wherein b is an integer ranging from 0 to 3 and c is an integer ranging from 1 to 20 and, for example, ranging from 2 to 12.

As disclosed herein, the group R may be chosen, for example, from hexamethylene, 4,4′-biphenylenemethane, 2,4- and/or 2,6-tolylene, 1,5-naphthylene, p-phenylene and methylene-4,4-bis-cyclohexyl groups and the divalent group derived from isophorone.

In another embodiment of the present disclosure, the polysiloxane segment P can correspond to the general formula (X) below: 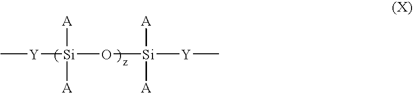
wherein:
the groups A, which may be identical or different, are chosen from C₁-C₂₀ monovalent hydrocarbon-based groups substantially free of ethylenic unsaturation and aromatic groups,
Y is a divalent hydrocarbon-based group, and
z is an integer chosen such that the average molecular mass of the polysiloxane segment ranges from 300 to 10,000.

The divalent group Y, as disclosed herein, may be chosen from the alkylene groups of formula —(CH₂)a-, wherein a is an integer that ranges from 1 to 10.

The groups A, as disclosed herein, may be chosen from C₁-C₁₈ alkyl groups, such as methyl, ethyl, propyl, isopropyl, butyl, pentyl, hexyl, octyl, decyl, dodecyl and octadecyl groups; cycloalkyl groups, for example the cyclohexyl group; aryl groups, for example phenyl and naphthyl; arylalkyl groups, for example benzyl and phenylethyl, and also tolyl and xylyl groups.

Non-limiting examples of fixing polyurethanes that may be mentioned include the dimethylolpropionic acid/isophorone diisocyanate/neopentyl glycol/polyester diols copolymer (also known under the name polyurethane-1, INCl name) sold under the brand name Luviset® Pur by the company BASF, and the dimethylolpropionid acid/isophorone diisocyanate/neopentyl glycol/polyester diols/silicone diamine copolymer (also known under the name polyurethane-6, INCl name) sold under the brand name Luviset® Si PUR A by the company BASF.

Another anionic polyurethane that may also be used is Avalure UR 450.

Additionally, polymers comprising sulfoisophthalate groups, such as the polymers AQ55 and AQ48 sold by the company Eastman, may also be used.

As disclosed herein, the anionic polymers can be chosen from acrylic acid copolymers such as the acrylic acid/ethyl acrylate/N-tert-butylacrylamide terpolymer sold under the name Ultrahold Strong® by the company BASF, and methacrylic acid/ethyl acrylate copolymers, for instance in aqueous dispersion, such as Luviflex Soft and Luvimer MAE sold by the company BASF. Crotonic acid-based copolymers may also be used, such as vinyl acetate/vinyl tert-butylbenzoate/crotonic acid terpolymers and crotonic acid/vinyl acetate/vinyl neododecanoate terpolymers sold under the name Resin 28-29-30 by the company National Starch, polymers derived from maleic, fumaric or itaconic acid or anhydride with vinyl esters, vinyl ethers, vinyl halides or phenylvinyl derivatives, acrylic acid and esters thereof, such as the methyl vinyl ether/monoesterified maleic anhydride copolymer sold under the name Gantrez® ES 425 by the company ISP, Luviset Si Pur, Mexomer PW, elastomeric or non-elastomeric anionic polyurethanes, polymers comprising sulfoisophthalate groups, anionic grafted silicone polymers, and also Amerhold DR 25 and VS 80.

As disclosed herein, the amphoteric polymers that can be used may be chosen from polymers comprising units B and C distributed randomly in the polymer chain, wherein B denotes a unit derived from a monomer comprising at least one basic nitrogen atom and C denotes a unit derived from an acid monomer comprising at least one carboxylic or sulfonic group, or alternatively B and C can be groups derived from carboxybetaine or sulfobetaine zwitterionic monomers; B and C can also be a cationic polymer chain comprising primary, secondary, tertiary or quaternary amine groups, wherein at least one of the amine groups bears a carboxylic or sulfonic group connected via a hydrocarbon group or alternatively B and C form part of a chain of a polymer comprising an ethylenedicarboxylic unit wherein one of the carboxylic groups has been made to react with a polyamine comprising at least one primary or secondary amine group.

In another embodiment of the present invention, the amphoteric polymers, as used herein, may be chosen from the following polymers:

(1) polymers resulting from the copolymerization of a monomer derived from a vinyl compound bearing a carboxylic group such as, for example, acrylic acid, methacrylic acid, maleic acid, x-chloroacrylic acid, and a basic monomer derived from a substituted vinyl compound containing at least one basic atom, such as dialkylaminoalkyl methacrylate and acrylate, dialkylaminoalkylmethacrylamides and -acrylamides. Such compounds are described, for instance, in U.S. Pat. No. 3,836,537.

The vinyl compound may also be a dialkyldiallylammonium salt such as diethyidiallylammonium chloride.

(2) polymers comprising units derived from:

a) at least one monomer chosen from acrylamides and methacrylamides substituted on the nitrogen with an alkyl group,

b) at least one acidic comonomer comprising at least one reactive carboxylic group, and

c) at least one basic comonomer such as acrylic and methacrylic acid esters comprising primary, secondary, tertiary and quaternary amine substituents, and the product of quaternization of dimethylaminoethyl methacrylate with dimethyl or diethyl sulfate.

As disclosed herein, the N-substituted acrylamides or methacrylamides may be, for example, compounds wherein the alkyl groups comprise from 2 to 12 carbon atoms such as N-ethylacrylamide, N-tert-butylacrylamide, N-tert-octylacrylamide, N-octylacrylamide, N-decylacrylamide, N-dodecylacrylamide and the corresponding methacrylamides.

As disclosed herein, the acidic comonomers can be chosen, for example, from acrylic acid, methacrylic acid, crotonic acid, itaconic acid, maleic acid and fumaric acid and alkyl monoesters, comprising 1 to 4 carbon atoms, or maleic or fumaric acids or anhydrides. As disclosed herein, the basic comonomers may be, for example, aminoethyl, butylaminoethyl, N,N′-dimethylaminoethyl and N-tert-butylaminoethyl methacrylates. The copolymers whose CTFA (4th edition, 1991) name is octylacrylamide/acrylates/butylaminoethyl methacrylate copolymer, such as the products sold under the name Amphomer® or Lovocryl® 47 by the company National Starch, may be used.

(3) crosslinked and alkylated polyamino amides partially or totally derived from polyamino amides of formula (XI): [symbol]
CO—R₄—CO—Z [symbol] (XI)
wherein R₄ is chosen from divalent groups derived from a saturated dicarboxylic acid, mono- and dicarboxylic aliphatic acids comprising an ethylenic double bond, esters of lower alkanols, comprising 1 to 6 carbon atoms, of these acids, or a group derived from the addition of any one of the said acids to a bis(primary) or bis(secondary) amine, and Z is a group derived from a bis(primary), mono- or bis(secondary) polyalkylene polyamine and can be chosen from:

a) in an amount ranging from 60 to 100 mol %, the group: 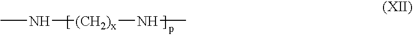
where x is equal to 2 and p is equal to 2 or 3, or alternatively x is equal to 3 and p is equal to 2
this group being derived from diethylenetriamine, from triethylenetetraamine or from dipropylenetriamine;

b) in an amount ranging from 0 to 40 mol %, the group (XII) above wherein x is equal to 2 and p is equal to 1 and which is derived from ethylenediamine, or the group derived from piperazine:

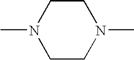

c) in an amount ranging from 0 to 20 mol %, the —NH(CH₂)₆—NH— group being derived from hexamethylenediamine,
these polyamino amides being crosslinked by addition reaction of a difunctional crosslinking agent chosen from epihalohydrins, diepoxides, dianhydrides and bis-unsaturated derivatives, using from 0.025 to 0.35 mol of crosslinking agent per amine group of the polyamino amide and alkylated by the action of acrylic acid, chloroacetic acid or an alkane sultone, or salts thereof.

The saturated carboxylic acids, as disclosed herein, may be chosen from acids comprising 6 to 10 carbon atoms, such as adipic acid, 2,2,4-trimethyladipic acid and 2,4,4-trimethyladipic acid, terephthalic acid, acids comprising an ethylenic double bond such as, for example, acrylic acid, methacrylic acid and itaconic acid. The alkane sultones used in the alkylation for example, may be propane sultone or butane sultone, the salts of the alkylating agents are for instance, the sodium or potassium salts.

(4) polymers comprising zwitterionic units of formula (XIII): 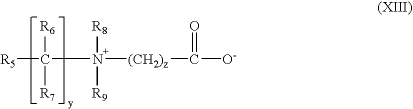
wherein R₅ denotes a polymerizable unsaturated group such as an acrylate, methacrylate, acrylamide or methacrylamide group, y and z each are an integer ranging from 1 to 3, R₆ and R₇ are chosen from a hydrogen atom, and methyl, ethyl and propyl groups, R₈ and R₉ are chosen from a hydrogen atom and alkyl groups such that the sum of the carbon atoms in R₁₀ and R₁₁ does not exceed 10.

The polymers comprising such units can also comprise units derived from nonzwitterionic monomers such as dimethyl- or diethylaminoethyl acrylate or methacrylate or alkyl acrylates or methacrylates, acrylamides or methacrylamides or vinyl acetate.

(5) polymers derived from chitosan comprising monomer units of the formulae (XIV), (XV), and (XVI): 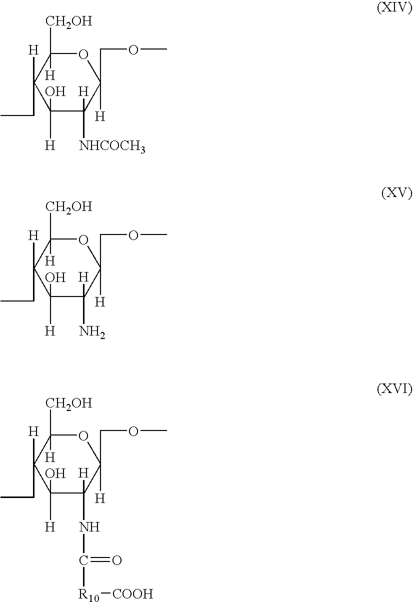
the unit (XIV) being present in an amount ranging from 0 to 30%, the unit (XV) in an amount ranging from 5% to 50% and the unit (XVI) in an amount ranging from 30% to 90%, it being understood that, in this unit, RI₀ is a group of formula: 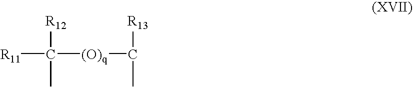
wherein, if q is equal to 0, R₁₁, R₁₂ and R₁₃, which may be identical or different, each are chosen from a hydrogen atom, and methyl, hydroxyl, acetoxy and amino residues, monoalkylamine residues, and dialkylamine residues that are optionally interrupted by at least one nitrogen atom and/or optionally substituted with at least one group chosen from amine, hydroxyl, carboxyl, alkylthio and sulfonic groups, and alkylthio residues, wherein. when the alkyl group bears an amino residue, at least one of the groups R₁₁, R₁₂ and R₁₃ being, in this case, a hydrogen atom;
or, if q is equal to 1, R₁₁, R₁₂ and R₁₃ are hydrogen atoms, as well as the salts formed by these compounds with bases or acids.

(6) polymers derived from the N-carboxyalkylation of chitosan.

(7) polymers of units of formula (XVIII) described, for example, in French Pat. No. 1 400 366: 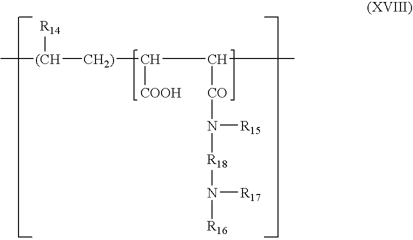
wherein R₁₄ is chosen from a hydrogen atom, and CH₃O, CH₃CH₂O and phenyl groups, R₁₅ is chosen from a hydrogen atom and lower alkyl groups such as methyl or ethyl, R₁₆ is chosen from a hydrogen atom and lower alkyl groups such as methyl or ethyl, R₁₇ is a lower alkyl group such as methyl or ethyl or a group corresponding to the formula: —R₁₈—N(R₁₆)₂, R₁₈ is chosen from a —CH₂—CH₂—, —CH₂—CH₂—CH₂— and —CH₂—CH(CH₃)— groups, R₁₆ having the meanings mentioned above,
and also the higher homologues of these groups, comprising up to 6 carbon atoms.

(8) amphoteric polymers of the type -D-X-D-X- chosen from:

a) polymers obtained by the action of chloroacetic acid or sodium chloroacetate on compounds comprising at least one unit of formula:
-D-X-D-X-D- (XIX)
where D is a group 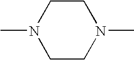
and X is the symbol E or E′; E or E′, which may be identical or different, are chosen from divalent groups that are alkylene groups with a straight or branched chain comprising up to 7 carbon atoms in the main chain, which is unsubstituted or substituted with hydroxyl groups and which may comprise, in addition to the oxygen, nitrogen and sulfur atoms, 1 to 3 aromatic and/or heterocyclic rings; the oxygen, nitrogen and sulfur atoms being present in the form of ether, thioether, sulfoxide, sulfone, sulfonium, alkylamine or alkenylamine groups, hydroxyl, benzylamine, amine oxide, quaternary ammonium, amide, imide, alcohol, ester and/or urethane groups.

b) polymers of formula:
-D-X-D-X- (XX)
where D is a group 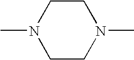
and X is the symbol E or E′ and at least once E′; wherein E has the meaning given above and E′ is a divalent group that is an alkylene group with a straight or branched chain comprising up to 7 carbon atoms in the main chain, which is unsubstituted or substituted with at least one hydroxyl group and comprising at least one nitrogen atom, the nitrogen atom being substituted with an alkyl chain that is optionally interrupted by an oxygen atom and necessarily comprising at least one carboxyl functional group or at least one hydroxyl functional group and betainized by reaction with chloroacetic acid or sodium chloroacetate.

(9) (C₁-C₅)alkyl vinyl ether/maleic anhydride copolymers partially modified by semiamidation with an N,N-dialkylaminoalkylamine such as N,N-dimethylamino-propylamine or by semiesterification with an N,N-dialkanolamine. These copolymers may also comprise other vinyl comonomers such as vinylcaprolactam.

In at least one embodiment of the present disclosure, the amphoteric fixing polymers that may be used in the process, as disclosed herein, may be chosen from branched block copolymers comprising:

(a) nonionic units derived from at least one monomer chosen from C₁-C₂₀ alkyl (meth)acrylates, N-mono(C₂-C₁₂ alkyl)(meth)acrylamides and N,N-di(C₂-C₁₂ alkyl)(meth)acrylamides,

(b) anionic units derived from at least one monomer chosen from acrylic acid and methacrylic acid, and

(c) polyfunctional units derived from at least one monomer comprising at least two polymerizable unsaturated functional groups,

and may have, for example, a structure comprising hydrophobic blocks onto which are fixed, via polyfunctional units (c), several more hydrophilic blocks.

The amphoteric polymers, as disclosed herein, have, for instance at least two glass transition temperatures (Tg), at least one of which is greater than 20° C. and the other is less than 20° C.

The amphoteric polymers, as disclosed herein, may be, for example, polymers comprising units derived from:

a) at least one monomer chosen from acrylamides and methacrylamides substituted on the nitrogen with an alkyl group,

b) at least one acidic comonomer comprising at least one reactive carboxylic groups, and

c) at least one basic comonomer such as acrylic and methacrylic acid esters comprising primary, secondary, tertiary and quaternary amine substituents, and the product of quaternization of dimethylaminoethyl methacrylate with dimethyl or diethyl sulfate.

Non-limiting mention may be made of the polymers sold under the name Amphomer by the company National Starch.

As disclosed herein, the medium for composition (B) comprising the fixing polymer is a cosmetically acceptable aqueous, alcoholic or aqueous-alcoholic medium. The cosmetically acceptable organic solvents may be, for example, monoalcohols, polyols or ethers of these alcohols or polyols. Additional non-limiting examples that may be mentioned include ethanol, isopropanol, glycerol, propylene glycol and propylene glycol monomethyl ether.

The at least one fixing polymer can be present in an amount ranging from 0.1% to 20% and for example an amount ranging from 1% to 15% by weight relative to the total weight of composition (B).

As disclosed herein, the shaping step of the process is a styling step during which the hair is given the desired shape by simply using the hands or by a simple mechanical device/tool for easily holding longer locks of hair in place, such as clips, combs, hair slides, tie-rolls and elastic bands. This setting in place may be performed by the user and does not require the intervention of a hair stylist. In any event, this step does not include the use of mechanical tensioning tools.

This step is performed at room temperature or with heating so as to bring the hair to a temperature ranging from 30 to 60° C.

This step is typically followed by a period of leave-on time ranging from 10 minutes to 25 minutes.

At this stage, it is then possible to perform rinsing. However, the user should take great care to ensure that this rinsing leaves an amount of precipitate sufficient to conserve the shape of the hairstyle during the fixing step.

Typically, the styling step is not followed by rinsing.

The fixing composition (C) may be applied to the hair after the leave-on time following shaping. This application takes place, of course, as far as is possible without changing the shape imposed on the head of hair in the preceding step. Such an application may take place, for example, using an aerosol device or a pump-dispenser bottle that dispenses a fixing mousse or fine droplets of a fixing solution. Application of the fixing composition in the form of mousse or of fine droplets allows softening of the precipitated polymer gradually during the fixing step. The nature of the oxidizing agent used in this fixing composition is not a deciding factor for the present disclosure, and any known oxidizing agent usually used for oxidizing the hair may be used, such as hydrogen peroxide, urea peroxide, bromates and persalts, and mixtures thereof.

The oxidizing agent, in one embodiment of the present disclosure, may be hydrogen peroxide.

The period of leave-on time for the fixing step ranges from 2 to 30 minutes, for instance ranging from 2 to 15 minutes and further for example ranging from 2 to 7 minutes.

The composition remaining on the hair is then removed by rinsing.

In at least one embodiment of the present disclosure, the fixing composition (C) comprises at least one surfactant, for example, an anionic surfactant.

The rinsing is optionally followed by washing and/or another hair treatment.

In, yet, another embodiment of the present disclosure, the reducing composition (A) comprises at least one cationic polymer and may also comprise a salt of a dithio acid such as ammonium dithioglycolate.

In another embodiment of the present disclosure, the step of applying a reducing composition (A) is preceded by a step of applying a composition (0) comprising at least one cationic polymer.

In performing the process, as disclosed herein, it is possible to follow the step of applying a cationic polymer by rinsing. The user should take care, however, to ensure that this rinsing leaves an amount of cationic polymer sufficient to preserve the properties provided by this polymer.

Typically, the step of applying at least one cationic polymer is not followed by rinsing.

Another aspect of the present disclosure also relates to a multi-compartment device for permanently reshaping the hair, comprising, in separate containers, the compositions used in the process as disclosed herein. When the compositions are contained in a multi-compartment device, the multi-compartment device may comprise a compartment (D1) comprising a composition comprising at least one cationic polymer.

As used herein, the term “cationic polymer” is understood to mean any polymer comprising cationic groups or groups that may be ionized into cationic groups.

In at least one embodiment of the present disclosure, the at least one cationic polymer may be chosen from those which comprise units comprising primary, secondary, tertiary and/or quaternary amine groups which can either form part of the main polymer chain or can be borne by a lateral substituent directly connected thereto.

The cationic polymers, as described herein, may have a number-average molecular mass ranging from 500 to 5×10⁶ and for example ranging from 10³ to 3×10⁶.

Further, among the cationic polymers that may be used, non-limiting mention may be made of polymers such as polyamines, polyaminoamides and polyquaternary ammoniums. These are known products.

One family of cationic polymers is the family of silicone cationic polymers. Among these polymers which may be used, non-limiting mention may be made of:

(a) the silicone polymers of formula (IV):
R⁶ₐG⁵₃₋ₐ—Si(OSiG⁶₂)ₙ—(OSiG⁷_{b}R⁷_{2-b})ₘ—O—SiG⁸₃₋ₐ—R⁸_{a′} (IV)
wherein:
G⁵, G⁶, G⁷ and G⁸, which may be identical or different, are chosen from a hydrogen atom, phenyl and OH groups, C₁-C₁₈ alkyl groups, for example methyl, C₂-C₁₈ alkenyl groups and C₁-C₁₈ alkoxy groups,
a and a′, which may be identical or different, is an integer ranging from 0 to 3, such as 0, b denotes 0 or 1, and such as 1,
m and n are integers such that the sum (n+m) may range from 1 to 2,000 and for example from 50 to 150, it being possible for n to be an integer ranging from 0 to 1999 and for example from 49 to 149, and for m to denote a integer ranging from 1 to 2000 and for example from 1 to 10;
R⁶, R⁷ and R⁸, which may be identical or different, are chosen from monovalent groups of formula —C_{q}H_{2q}OₛR⁹ₜL wherein q is a integer ranging from 1 to 8, s and t, which may be identical or different, are equal to 0 or 1, R⁹ is an optionally hydroxylated alkylene group and L is an optionally quaternized amino group chosen from the groups:
—NR″—CH₂—CH₂—N′(R″)₂
—N(R″)₂
—N^{⊕}(R″)₃A⁻
—N^{⊕}H(R″)₂A⁻
—N^{⊕}H₂(R″)A⁻
—N(R″)—CH₂—CH₂—N^{⊕}R″H₂A⁻
   wherein R″ can be chosen from hydrogen, phenyl, benzyl and monovalent saturated hydrocarbon-based groups, for example an alkyl group comprising from 1 to 20 carbon atoms, and A⁻ is a halide ion such as, for example, fluoride, chloride, bromide or iodide.

Products corresponding to this definition are, for example, the polysiloxanes referred to in the CTFA dictionary as “amodimethicone” or “trimethylsilylamodimethicone”.

A commercial product corresponding to this definition is a mixture (90/10 by weight) of a polydimethylsiloxane containing aminoethyl aminoisobutyl groups and of a polydimethylsiloxane sold under the name Q2-8220 by the company Dow Corning.

Such polymers are described, for example, in European Patent Application No.95238.

Non-limiting mention may be made of polymers of formula (IV) such as amino silicones comprising alkoxy groups, for instance the silicone Wacker Belsil ADM LO 61.

Other example polymers corresponding to formula (IV) are the silicone polymers of formula (VI):
T-(CH₂)ₛ—Si—[(OSiR⁵R⁶)ₜ—R⁷]_{y} (VI)
wherein:

R₁₀ is a monovalent hydrocarbon-based group comprising from 1 to 18 carbon atoms, and for instance a C₁-C₁₈ alkyl or C₂-C₁₈ alkenyl group, for example methyl;

R₁₁ is a divalent hydrocarbon-based group, for instance a C₁-C₁₈ alkylene group or a divalent C₁-C₁₈, for example C₁-C₈, alkylenoxy group;

Q⁻ is a halide ion, such as chloride;

r is an average statistical value ranging from 2 to 20 and for example ranging from 2 to 8;

s is an average statistical value ranging from 20 to 200 and for example ranging from 20 to 50.

Such polymers are described for instance in U.S. Pat. No. 4,185,087.

(b) the compounds of formula:
NH—[(CH₂)₃—Si[OSi(CH₃)₃]]₃
corresponding to the CTFA name “aminobispropyldimethicone”.

A non-limiting example of a polymer included within this category is the polymer sold by the company Union Carbide under the name “Ucar Silicone ALE 56”.

When these silicone polymers are used, at least one embodiment of the present disclosure is their joint use with cationic and/or nonionic surfactants. It is possible, for example, to use the product sold under the name “Cationic Emulsion DC 939” by the company Dow Corning, which comprises, besides amodimethicone, cationic and nonionic surfactants.

Another commercial product that may be used according to the present disclosure is the product sold under the name “Dow Corning Q2 7224” by the company Dow Corning comprising, in combination, a trimethylsilylamodimethicone and nonionic surfactants.

In accordance with the present disclosure, the polymers of polyamine, polyaminoamide and polyquaternary ammonium type may be used are, for example, those described in French Patents Nos. 2 505 348 or 2 542 997. Among these polymers, non-limiting mention may be made of:

(1) Quaternized or non-quaternized vinylpyrrolidone/dialkylaminoalkyl acrylate or methacrylate copolymers, such as the products sold under the name “Gafquat®” by the company ISP, such as, for example, Gafquat 734, 755 or HS100, or alternatively the product known as “Copolymer 937”. These polymers are described in detail, for example, in French Pat. Nos. 2 077 143 and 2 393 573.

(2) Cellulose ether derivatives comprising quaternary ammonium groups described, for example, in French Patent No.1 492 597, and for example the polymers sold under the names “JR” (JR 400, JR 125, JR 30M) or “LR” (LR 400, LR 30M) by the company Union Carbide Corporation. These polymers are also defined in the CTFA dictionary as quaternary ammoniums of hydroxyethylcellulose which has reacted with an epoxide substituted with a trimethylammonium group.

(3) Cationic cellulose derivatives such as cellulose copolymers or cellulose derivatives grafted with a water-soluble quaternary ammonium monomer, and described for example in U.S. Pat. No. 4,131,576, such as hydroxyalkylcelluloses, for example hydroxymethyl-, hydroxyethyl- or hydroxypropylcelluloses grafted for instance with a methacryloylethyltrimethylammonium, methacrylamidopropyltrimethylammonium or dimethyldiallylammonium salt.

The commercial products corresponding to this definition are, for instance, the products sold under the names “Celquat L 200” and “Celquat H 100” by the company National Starch.

(4) The cationic guar gums described, for example, in U.S. Pat. Nos. 3,589,578 and 4,031,307 and, for example, the product sold under the name “Jaguar C13 S” sold by the company Meyhall.

(5) Polymers comprising piperazinyl units and of divalent alkylene or hydroxyalkylene groups comprising straight or branched chains, optionally interrupted with oxygen, sulfur or nitrogen atoms or with aromatic or heterocyclic rings, as well as the oxidation and/or quaternization products of these polymers. Such polymers are described for instance in French Patent Nos. 2 162 025 and 2 280 361.

(6) Water-soluble polyaminoamides prepared for instance by polycondensation of an acidic compound with a polyamine; these polyaminoamides may be crosslinked with an epihalohydrin, a diepoxide, a dianhydride, an unsaturated dianhydride, a bis-unsaturated derivative, a bis-halohydrin, a bis-azetidinium, a bis-haloacyidiamine, a bis-alkyl halide or with an oligomer resulting from the reaction of a difunctional compound which is reactive with respect to a bis-halohydrin, a bis-azetidinium, a bis-haloacyldiamine, a bis-alkyl halide, an epihalohydrin, a diepoxide or a bis-unsaturated derivative, the crosslinking agent being used in an amount ranging from 0.025 to 0.35 mol per amine group of the polyaminoamide; these polyaminoamides may be alkylated or, if they comprise at least one tertiary amine functional group, may be quaternized. Such polymers are described for example in French Pat. Nos. 2 252 840 and 2 368 508.

(7) Polyaminoamide derivatives resulting from the condensation of polyalkylene polyamines with polycarboxylic acids, followed by an alkylation with difunctional agents. Non-limiting mention may be made, for example, of adipic acid/dialkylaminohydroxyalkyl-dialkylenetriamine polymers wherein the alkyl group comprises from 1 to 4 carbon atoms and for example denotes methyl, ethyl or propyl. Such polymers are described for instance in French Patent No.1 583 363.

Among these derivatives, non-limiting mention may be made of the adipic acid/dimethylaminohydroxypropyl/diethylenetriamine polymers sold under the name “Cartaretine F, F4 or F8” by the company Sandoz.

(8) Polymers obtained by reacting a polyalkylene polyamine comprising two primary amine groups and at least one secondary amine group with a dicarboxylic acid chosen from diglycolic acid and saturated aliphatic dicarboxylic acids comprising from 3 to 8 carbon atoms. The molar ratio between the polyalkylene polyamine and the dicarboxylic acid ranges from 0.8:1 to 1.4:1, the polyaminoamide resulting therefrom being made to react with epichlorohydrin in a molar ratio of epichlorohydrin relative to the secondary amine group in the polyaminoamide ranging from 0.5:1 to 1.8:1. Such polymers are described for example in U.S. Pat. Nos. 3,227,615 and 2,961,347.

Polymers of this type are sold, for example, under the name “Hercosett 57” by the company Hercules Inc. or alternatively under the name “PD 170” or “Delsette 101” by the company Hercules in the case of the adipic acid/epoxypropyl/diethylenetriamine copolymer.

(9) Cyclopolymers of methyidiallylamine or of diallyldimethylammonium, such as homopolymers or copolymers comprising, as main constituent of the chain, units corresponding to formulae (VIII) or (VIII′):

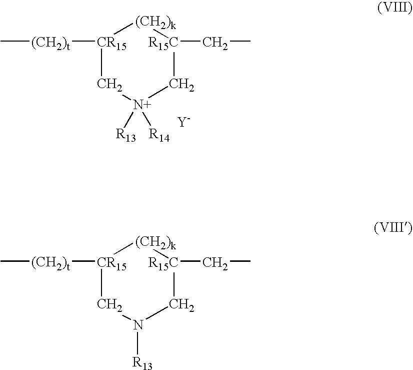

wherein formulae k and t are equal to 0 or 1, the sum k+t being equal to 1; R₁₅ is a hydrogen atom or a methyl group; R₁₃ and R₁₄, independently of each other, are chosen from alkyl groups comprising from 1 to 8 carbon atoms, hydroxyalkyl groups wherein the alkyl group, for example, comprises 1 to 5 carbon atoms, and lower amidoalkyl groups, or R₁₃ and R₁₄ may form, together with the nitrogen atom to which they are attached, heterocyclic groups such as piperidyl or morpholinyl; Y⁻ is an anion such as bromide, chloride, acetate, borate, citrate, tartrate, bisulfate, bisulfite, sulfate or phosphate. These polymers are described, for example, in French Patent No. 2 080 759 and in its Certificate of Addition No. 2 190 406.

Non-limiting mention may be made, for example, of the diallyldimethylammonium chloride homopolymer sold under the name “Merquat 100” by the company Merck and the copolymers of diallyldimethylammonium chloride and of acrylamide sold under the name “Merquat 550”.

(10) The diquaternary ammonium polymer comprising repeating units of formula (XIII): 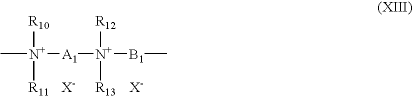
wherein: R₁₀, R₁₁, R₁₂ and R₁₃, which may be identical or different, are chosen from aliphatic, alicyclic and arylaliphatic groups comprising from 1 to 20 carbon atoms, and lower hydroxyalkyl aliphatic groups, or R₁₀, R₁₁, R₁₂ and R₁₃, together or separately, constitute, with the nitrogen atoms to which they are attached, heterocycles optionally comprising a second hetero atom other than nitrogen, or alternatively R₁₀, R₁₁, R₁₂ and R₁₃ are chosen from linear and branched C₁-C₆ alkyl groups substituted with a nitrile, ester, acyl, amide or —CO—O—R₂₀-D or —CO—NH—R₂₀-D group wherein R₂₀ is an alkylene and D is a quaternary ammonium group;
A₁ and B₁ may be chosen from polymethylenic groups comprising from 2 to 20 carbon atoms which can be linear or branched, saturated or unsaturated and which may comprise, linked to or intercalated in the main chain, at least one aromatic ring, at least one oxygen or sulfur atoms or sulfoxide, sulfone, disulfide, amino, alkylamino, hydroxyl, quaternary ammonium, ureido, amide or ester groups, and
X⁻ is an anion derived from an inorganic or organic acid;
A₁, R₁₀ and R₁₂ may form, with the two nitrogen atoms to which they are attached, a piperazine ring; in addition, if A₁ is a linear or branched, saturated or unsaturated alkylene or hydroxyalkylene group, B₁ may also denote a group (CH2)ₙ—CO-D-OC—(CH₂)ₙ— wherein D is:

a) a glycol residue of formula: —O—Z—O—, wherein Z is a linear or branched hydrocarbon-based group or a group corresponding to one of the following formulae:
—(CH₂—CH₂—O)ₓ—CH₂—CH₂—
—[CH₂—CH(CH₃)—O]_{y}—CH₂—CH(CH₃)—
wherein x and y are each an integer ranging from 1 to 4, representing a defined and unique degree of polymerization or any number from 1 to 4 representing an average degree of polymerization;

b) a bis-secondary diamine residue such as a piperazine derivative;

c) a bis-primary diamine residue of formula: —NH—Y—NH— wherein Y is a linear or branched hydrocarbon-based group, or alternatively the divalent group —CH₂—CH₂—S—S—CH₂—CH₂—;

d) a ureylene group of formula: —NH—CO—NH—.

X⁻ is, for example, an anion such as chloride or bromide.

These polymers have a number-average molecular mass, for example, ranging from 1,000 to 100,000.

Polymers of this type are described, for example, in French Patent Nos. 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3929,990, 3,966,904, 4,005,193, 4,025,617, 4,025,627, 4,025,653, 4,026,945 and 4,027,020.

In, at least, one embodiment of the present disclosure, it is possible to use polymers that comprise repeating units of formula (XIV):

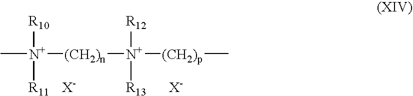

wherein R₁₀, R₁₁, R₁₂ and R₁₃, which may be identical or different, are chosen from alkyl and hydroxyalkyl groups comprising 1 to 4 carbon atoms, n and p are integers ranging from 2 to 20, and X⁻ is an anion derived from an inorganic or organic acid.

In another embodiment of the present disclosure, one compound that may be used is the one for which R₁₀, R₁₁, R₁₂ and R₁₃ are methyl groups and n=3, p=6 and X⁻ is a chloride ion, which is known as Hexadimethrine chloride according to the INCl (CTFA) nomenclature, such as Mexomer® PO from the company Chimex.

(11) Polymers comprising polyquaternary ammonium repeating units, for example comprising units of formula (X): 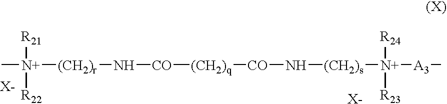
wherein:
R₂₁, R₂₂, R₂₃ and R₂₄, which may be identical or different, are chosen from hydrogen atoms and methyl, ethyl, propyl, β-hydroxyethyl, β-hydroxypropyl and —CH₂CH₂(OCH₂CH₂)ₚOH groups,
wherein p is an integer ranging from 0 to 6, with the proviso that R₂₁, R₂₂, R₂₃ and R₂₄ are not simultaneously hydrogen atoms,
r and s, which may be identical or different, are integers ranging from 1 to 6,
q is an integer ranging from 0 to 34,
X is a halogen atom,
A₃ is a dihalide group or —CH₂—CH₂—O—CH₂—CH₂—.

Such compounds are described for example in European Patent Application No. 122 324.

Non-limiting mention may be made of the following products:

Mirapol® A 15”, “Mirapol® AD1”, “Mirapol® AZ1” and “Mirapol® 175” sold by the company Miranol.

(12) Homopolymers or copolymers derived from acrylic or methacrylic acids and comprising units:

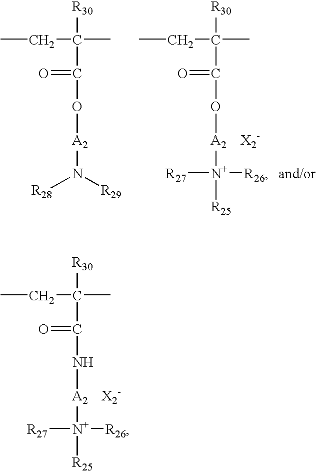

wherein the groups R₃₀ independently are H or CH₃, the groups A₂ independently are a linear or branched alkyl group ranging from 1 to 6 carbon atoms or a hydroxyalkyl group ranging from 1 to 4 carbon atoms, the groups R₂₅, R₂₆ and R₂₇, which may be identical or different, independently are alkyl groups ranging from 1 to 18 carbon atoms or a benzyl group,
the groups R₂₈ and R₂₉ are hydrogen atoms or alkyl groups ranging from 1 to 6 carbon atoms,

X₂⁻ is an anion, for example methosulfate or halide, such as chloride or bromide.

The comonomer(s) that can be used to prepare the corresponding copolymers belong to the family of acrylamides, methacrylamides, diacetone acrylamides and acrylamides and methacrylamides substituted on the nitrogen with lower alkyls, alkyl esters, acrylic or methacrylic acids, vinylpyrrolidone or vinyl esters.

(13) Quaternary polymers of vinylpyrrolidone and of vinylimidazole such as, for example, the products sold under the names Luviquat® FC 905, FC 550 and FC 370 by the company BASF.

(14) Polyamines such as Polyquart H sold by Henkel, referred to under the name “Polyethylene glycol (15) tallow polyamine” in the CTFA dictionary.

(15) Crosslinked methacryloyloxyethyltrimethylammonium chloride polymers such as the polymers obtained by homopolymerization of dimethylaminoethyl methacrylate quaternized with methyl chloride, or by copolymerization of acrylamide with dimethylaminoethyl methacrylate quaternized with methyl chloride, the homo- or copolymerization being followed by crosslinking with a compound comprising olefinic unsaturation, for instance methylenebisacrylamide. An acrylamide/methacryl-oyloxyethyltrimethylammonium chloride crosslinked copolymer (20/80 by weight) in the form of a dispersion comprising 50% by weight of the said copolymer in mineral oil, for example, may be used. This dispersion is sold under the name Salcare SC 92 by the company Allied Colloids. A methacryloyloxyethyltrimethylammonium chloride crosslinked homopolymer comprising about 50% by weight of the homopolymer in mineral oil may also be used. This dispersion is sold under the name Salcare® SC 95 by the company Allied Colloids.

Other cationic polymers which may be used in the context of the disclosure are polyalkyleneimines, such as polyethyleneimines, polymers comprising vinylpyridine or vinylpyridinium units, condensates of polyamines and of epichlorohydrin, polyquaternary ureylenes and chitin derivatives.

Among all the cationic polymers that may be used in the context of the present disclosure, further non-limiting examples may be made of cationic polymers such as dimethyldiallylammonium chloride homopolymer sold under the name Merquat® 100 by the company Merck, and the diquaternary ammonium polymers of formula (IX) or of formula (X).

According to the present disclosure, the at least one cationic polymer may be present in an amount ranging from 0.01% to 20% by weight, for instance ranging from 0.1% to 15% by weight, such as ranging from 0.5% to 5% by weight relative to the total weight of composition (O) or of composition (A).

Each of the compositions of the multi-compartment device may also comprise at least one active principle and/or cosmetic adjuvant commonly used in haircare. These additives may be chosen, for example, from vitamins, amino acids, oligopeptides, peptides, hydrolysed and non-hydrolysed, modified and unmodified proteins, enzymes, branched and unbranched fatty acids and fatty alcohols, animal, plant and mineral waxes, ceramides and pseudoceramides, hydroxylated organic acids, UV-screening agents, antioxidants and free-radical scavengers, chelating agents, anti dandruff agents, seborrhoea regulators, soothing agents, cationic, anionic, nonionic and amphoteric surfactants, nonionic polymers, nonionic silicones, mineral, plant and animal oils, polyisobutenes and poly(x-olefins), fatty esters, and hair dyes such as direct dyes and pigments.

Needless to say, a person skilled in the art will take care to select this or these additional active principles and/or cosmetic adjuvants such that the beneficial properties intrinsically associated with the device and process in accordance with the present disclosure are not, or are not substantially, adversely affected by the envisaged addition(s).

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific example are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

The following compositions in accordance with the present disclosure were prepared:

Reducing composition (A)

| | | |
|---|---|---|
| Thioglycolic acid | 5.0 | g |
| Aqueous ammonia | 6.0 | g |
| Ammonium chloride | 5.0 | g |
| Hydroxypropyl guar | 1.0 | g |
| Mexomer ® PO* (Chimex) | 1.5 | g |
| Tegobetaine ® HS** (Goldschmidt) | 1.5 | g |
| Fragrance, peptizer qs | | |
| Demineralized water qs | 100 | g |

*polymer of N,N,N,N-tetramethylhexamethylenediamine and of trimethylene chloride (INCI: Hexadimethrine chloride)
**cocoamidopropylbetaine and glyceryl laurate

Composition (B)

| | | |
|---|---|---|
| Luviset Si-Pur | 5.13 | g |
| AQ 48 | 6.15 | g |
| Fragrance | 0.40 | g |
| Alcohol | 28.95 | g |
| Demineralized water qs | 100 | g |

An aerosol comprising 65 g of composition (B) and 35 g of dimethyl ether propellant was prepared.

Fixing composition (C)

| | | |
|---|---|---|
| 50% hydrogen peroxide solution | 4.8 | g |
| Stabilizers | 0.03 | g |
| Ammonium lauryl sulfate | 4.0 | g |
| Fragrance, peptizer qs | | |
| Demineralized water qs | 100 | g |

These three compositions were applied in accordance with the process according to the disclosure to half-heads.

For comparative purposes, the other half of each half-head was simply rinsed with water. The hair of the half-heads treated according to process in accordance with the present disclosure was judged to be more styleable and the head of hair was judged to be more voluminous: the process allowed better styling, while providing texture and body.

When the hair of the half-heads is dyed hair, it was observed that the coloration was respected and that these effects were remnant over time.

What is claimed is:

1. A process for permanently reshaping keratin fibers comprising, in the following order:
(a) applying to the keratin fibers a reducing composition (A) comprising at least one keratin fiber reducing agent and at least one salt chosen from organic and mineral salts,
(b) shaping of the keratin fibers by styling and applying to the keratin fibers a composition (B) comprising at least one fixing polymer chosen from anionic and amphoteric fixing polymers, in an aqueous, alcoholic or aqueous-alcoholic medium,
(c) after a period of leave-on time, applying to the keratin fibers a fixing composition (C) comprising at least one oxidizing agent,
(d) after a leave-on time, rinsing the keratin fibers;
wherein the sub-steps of shaping of the keratin fibers by styling and applying to the keratin fibers a composition (B) of step (b) can be performed in one of the following orders: shaping of the keratin fibers by styling, followed by applying to the keratin fibers a composition (B); applying to the keratin fibers a composition (B), followed by shaping of the keratin fibers by styling; or shaping of the keratin fibers by styling and simultaneously applying to the keratin fibers a composition (B).

2. A process according to claim 1, wherein step (b) is followed by rinsing.

3. A permanent reshaping process according to claim 1, wherein the at least one salt chosen from organic and mineral salts is chosen from ammonium salts and alkali metal salts.

4. A permanent reshaping process according to claim 3, wherein the ammonium salts and/or the alkali metal salts are provided by the reducing agent.

5. A permanent reshaping process according to claim 1, wherein the at least one salt chosen from organic and mineral salts is present in the reducing composition at a concentration greater than or equal to 0.3 M.

6. A permanent reshaping process according to claim 5 wherein the at least one salt is present in the reducing composition at a concentration ranging from 0.5 to 6M.

7. A permanent reshaping process according to claim 5, wherein the at least one salt chosen from organic and mineral salts is chosen from sodium chloride, magnesium chloride, ammonium carbonate and ammonium citrate.

8. A permanent reshaping process according to claim 1, wherein the at least one keratin fiber reducing agent is chosen from sulfites, bisulfites and thiols.

9. A permanent reshaping process according to claim 1, wherein the at least one keratin fiber reducing agent is chosen from cysteine, cysteamine, thiolactic acid and thioglycolic acid and cosmetically acceptable salts thereof.

10. A permanent reshaping process according to claim 9, wherein the at least one keratin fiber reducing agent is chosen from ammonium thioglycolate.

11. A permanent reshaping process according to claim 1, wherein the at least one keratin fiber reducing agent in the reducing composition (a) is present in an amount ranging from 1% to 25% by weight with respect to the total weight of the composition.

12. A permanent reshaping process according to claim 11, wherein the at least one keratin fiber reducing agent in the reducing composition (a) is present in an amount ranging from 1% to 10% by weight, with respect to the total weight of the composition.

13. A permanent reshaping process according to claim 1, wherein the at least one anionic fixing polymer is chosen from acrylic acid copolymers; crotonic acid-based copolymers; copolymers derived from maleic, fumaric and/or itaconic acid and/or anhydride with vinyl esters, vinyl ethers, vinyl halides, phenylvinyl derivatives and/or acrylic acid and esters thereof; elastomeric and non-elastomeric anionic polyurethanes; polymers comprising sulfoisophthalate groups and anionic grafted silicone polymers.

14. A permanent reshaping process according to claim 1, wherein the at least one amphoteric fixing polymer is chosen from copolymers comprising units derived from:
at least one monomer chosen from acrylamides and methacrylamides substituted on the nitrogen with an alkyl group,
at least one acidic comonomer comprising at least one reactive carboxylic group, and
at least one basic comonomer comprising primary, secondary, tertiary and quaternary amine substituents, and the product of quaternization of dimethylaminoethyl methacrylate with dimethyl or diethyl sulfate.

15. A permanent reshaping process according to claim 14, wherein the at least one basic comonomer can be chosen from acrylic and methacrylic acid esters.

16. A permanent reshaping process according to claim 1, wherein the at least one keratin fiber fixing polymer is present in an amount ranging from 0.1% to 20% by weight relative to the total weight of composition (B).

17. A permanent reshaping process according to claim 16, wherein the at least one keratin fiber fixing polymer is present in an amount ranging from 1% to 15% by weight relative to the total weight of composition (B).

18. A permanent reshaping process according to claim 1, wherein the at least one oxidizing agent is chosen from hydrogen peroxide, urea peroxide, bromates and persalts.

19. A permanent reshaping process according to claim 1, wherein the period of leave-on time for the fixing step ranges from 2 to 30 minutes.

20. A permanent reshaping process according to claim 19, wherein the period of leave-on time for the fixing step ranges from 2 to 7 minutes.

21. A permanent reshaping process according to claim 1, wherein the fixing composition (B) comprises at least one surfactant.

22. A permanent reshaping process according to claim 1, wherein the reducing composition (A) comprises at least one cationic polymer.

23. A permanent reshaping process according to claim 1, wherein the application of a reducing composition (A) is preceded by the application of a composition comprising at least one cationic polymer.

24. A permanent reshaping process according to claim 23, wherein the application of a composition comprising at least one cationic polymer is followed by rinsing.

25. A permanent reshaping process according to claim 22, wherein the at least one cationic polymer is chosen from quaternary cellulose ether derivatives, copolymers of cellulose with a water-soluble quaternary ammonium monomer, cyclopolymers, cationic polysaccharides, cationic silicone polymers, quaternized and non-quaternized vinylpyrrolidone/dialkylaminoalkyl acrylate and methacrylate copolymers, quaternary polymers of vinylpyrrolidone and of vinylimidazole, polyamidoamines, and polymers comprising polyquaternary ammonium repeating units.

26. A multi-compartment device for permanently reshaping hair comprising at least:
a first compartment containing a reducing composition (A) comprising at least one keratin fiber reducing agent and at least one salt chosen from organic and mineral salts,
a second compartment containing a composition (B) comprising at least one fixing polymer chosen from anionic and amphoteric fixing polymers, in an aqueous, alcoholic or aqueous-alcoholic medium, and
a third compartment containing a fixing composition (C) comprising at least one oxidizing agent.
